(19) United States
(12) Patent Application Publication
(10) Pub. No.: US 2023/0026559 A1
(43) Pub. Date: Jan. 26, 2023

(54) ANALYSIS OF CELL SIGNATURES FOR DISEASE DETECTION

(71) Applicant: NOVIGENIX SA, Epalinges (CH)

(72) Inventors: Sahar HOSSEINIAN EHRENSBERGER, Epalinges (CH); Laura CIARLONI, Epalinges (CH); Sylvain MONNIER-BENOIT, Epalinges (CH); Jan GROEN, Epalinges (CH); Victoria WOSIKA, Epalinges (CH)

(21) Appl. No.: 17/784,019

(22) PCT Filed: Dec. 10, 2020
(86) PCT No.: PCT/EP2020/085586

§ 371 Date: Jun. 9, 2022

(30) Foreign Application Priority Data
Dec. 10, 2019 (EP) 19215017.5

(51) Int. Cl. C12Q 1/6886; B01L 3/00

(52) U.S. Cl. CPC C12Q 1/6886; B01L 3/50; C12Q 2600/158

(57) ABSTRACT

The present invention relates to methods for determining biomarker signatures that are relevant for detecting a disease in a patient or identifying altered abundance of cells within the patient. Also disclosed are methods for detecting a disease or altered cell type abundance in a patient by measuring said biomarker signature for at least one cell type.

### CROSS REFERENCE TO RELATED APPLICATIONS

This application claims priority to EP19215017.5, filed Dec. 10, 2019, the disclosure of which application is hereby incorporated by reference.

### TECHNICAL FIELD

The present invention relates to methods for determining biomarker signatures that are relevant for detecting a disease in a patient or identifying altered abundance of cells within the patient. Also disclosed are methods for detecting a disease or altered cell type abundance in a patient by measuring said biomarker signature for at least one cell type.

### BACKGROUND

Colorectal Cancer (CRC) is the second leading cause of cancer mortality worldwide. Effective and non-invasive biomarkers are needed to improve early diagnosis and disease management.

Immune checkpoint inhibitors (ICIs) such as anti-PD1 have become one of the main treatments for patients with metastatic bladder cancer (BC). Predictive biomarkers in BC are an unmet need, with only a minority of patients (20%) showing benefit from ICIs. Immune cells play a key role in tumor progression.

Circulating immune cell count is a potential cancer biomarker, as indicated for instance by the association of high blood neutrophil-to-lymphocyte ratio with poor prognosis in patients with cancer.

Various approaches exist for counting cells, especially for circulating immune cells. For instance, cells can be counted manually using a counting chamber or using immunohistochemistry techniques but these methods are very time consuming. There are also many automated direct cell counting systems notably flow cytometry, but these methods are generally expensive.

Moreover, these direct counting methods need to be performed at the time the biological sample is taken, or to process the biological sample with a specific protocol. Unfortunately, these direct cell counting methods to quantify the number of cells are rarely performed for samples analyzed at the gene expression level.

To fill this gap, diverse computational methods have been developed to estimate the cell abundance, in particular immune cell fractions, in a tissue, in particular tumor tissue or blood, from bulk gene expression data when direct counting of cells is not available. These methods are referred to as deconvolution methods.

For instance, Racle et al. developed a new computer-based tool (EPIC) that accurately estimate the fraction of tumor and immune cell types from bulk tumor gene expression data. (“Simultaneous enumeration of cancer and immune cell types from bulk tumor gene expression data”, Elife. 2017 November 13; 6.).

Racle et al optimized their approach to estimate the abundance of infiltrating immune cells from the solid tumor in which they infiltrate. However, they do not teach the use of these biomarkers for the detection of said solid tumors nor they optimized the gene signatures for blood and circulating immune cells.

Therefore, there is a need for alternative methods to facilitate cell abundance measurements or estimation for the detection of diseases from gene expression data.

### SUMMARY OF THE INVENTION

This object has been achieved by providing a method for detecting a disease in a subject by estimating the abundance of at least one cell type in a subject's test sample, the method comprising:

i) determining at least one cell type relevant for the detection of said disease;

ii) providing a biomarker signature for said cell type, said biomarker signature comprising at least one gene whose expression is associated with the abundance of said cell type;

iii) computing a cell signature score corresponding to a level of expression of said at least one gene in the biomarker signature in the test sample; and

iv) comparing the cell signature score with a reference value to deduce if the subject is suffering, or not, from said disease.

A further object of the present invention is to provide a method for determining the progression or regression of a disease in a subject suffering therefrom, said method comprising:

i) computing a cell signature score corresponding to a level of expression of at least one gene in the biomarker signature in a test sample obtained form said subject; and

ii) periodically comparing the cell signature score with a reference value or with the cell signature score determined previously,

wherein an alteration in the cell signature score associated with the abundance of at least one cell type in said biological sample, relative to the reference value or the cell signature score determined previously, is indicative of the progression or regression of said disease.

A further object of the present invention is to provide a method of stratifying a disease in a subject suffering therefrom, said method comprising:

i) providing a biomarker signature for a cell type relevant for the detection of said disease, said biomarker signature comprising at least one gene whose expression is associated with the abundance of said cell type;

ii) computing a cell signature score corresponding to a level of expression of said at least one gene in the biomarker signature in the test sample; and

iii) comparing the cell signature score with a reference value,

wherein a cell signature score superior or inferior to the reference value is indicative of the disease stage or grade.

A further object of the present invention is to provide a method for determining if a subject suffering from a disease is responsive to a treatment, said method comprising

i) computing a cell signature score corresponding to a level of expression of at least one gene in the biomarker signature in a test sample obtained from said subject, and

ii) periodically comparing the cell signature score with a reference value or with the cell signature score determined previously,

wherein an alteration in the cell signature score associated with the abundance of at least one cell type in said biological sample, relative to the reference value or the cell signature score determined previously, is indicative of the responsiveness of the subject to the treatment.

Also provided is a device for performing a method according to any one of the preceding claims, said device comprising:

i) a sample chamber for a test sample collected from a subject;

ii) an assay module in fluid communication with said sample chamber, said assay module comprising means and/or reagents for detecting and/or measuring, directly or indirectly, the gene expression in said test sample;

iii) means for computing a cell signature score; and

iv) a user interface wherein said user interface relates the cell signature score to detecting a disease in said subject, stratifying a disease or determining the responsiveness to a treatment.

Also provided is a method to identify at least one gene expression signature highly specific for a given cell type, the method comprising:

i) compiling a repertoire of candidate genes for said cell type from, e.g., previously published consensus signatures and/or public databases,

ii) filtering the candidate gene repertoire for lowly expressed and highly variable genes by comparing the expression levels in the organ of interest, setting a threshold to retain the reliably measurable genes,

iii) clustering the genes based on their correlation on at least three public and/or private datasets and selecting highly correlated gene clusters, in each dataset,

iv) confirming the specificity of the selected gene clusters of each dataset by functional analysis,

v) identifying a core gene signature defined as the gene overlap among the gene clusters selected in each dataset, and
vi) validating the specificity of the gene signature for the target cell type on an independent gene expression dataset derived from the purified or enriched target cell type.

Further provided is the use of at least one gene of a cell specific signature in a method or device of the invention.

### DESCRIPTION OF THE FIGURES

FIG. 1: Boxplots of B cells, T cells, NK cells, monocytes and neutrophils signature score (median expression levels) in the control (CON), and Colorectal Cancer (CRC). Immune cell signature scores are calculated on PBMC gene expression data generated by RNA-Seq

FIG. 2: Boxplots of B cells, T cells, NK cells, monocytes and neutrophils signature score (median expression levels) from whole blood of bladder cancer patients treated with anti-PD1. Signature levels were compared in treatment responders and non-responders (A) at baseline before treatment, and (B) during treatment. Immune cell signature scores are calculated on whole blood gene expression data generated by RNA-Seq

FIG. 3: Specificity testing of the cell signatures on purified cell populations from the Monaco's RNA-Seq dataset (A, B, C, D & E). Boxplot of cell signature scores (gene expression median) across different purified immune cell types and across different replicates per immune cell type. B: B cell; T: T cell; NK: natural killer cell; TFH: T follicular helper; Treg: T regulatory; Th: T helper; CE: central memory; EM: effector memory; TE: terminal effector; MAIT: mucosal-associated invariant T; SM: switched memory; NSM: non-switched memory; Ex: exhausted; LD: low-density; C: classical; I: intermediate; NC: non-classical; mDC: myeloid dendritic cells; pDC: plasmacytoid dendritic cells.

FIG. 4: Boxplot of B cells, T cells, NK, monocytes and neutrophils signature score (median expression levels) showing the discrimination of Tuberculosis patients from healthy controls (CON). Immune cell signature scores are calculated on whole blood gene expression data generated by RNA-Seq.

### DETAILED DESCRIPTION OF THE INVENTION

The above problems are solved or at least minimized by the methods according to present invention.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of “include/including”, that is to say permitting the presence of one or more features or components.

As used herein, the singular form “a”, “an” and “the” include plural references unless the context clearly dictates otherwise.

As used herein, “at least one” means “one or more”, “two or more”, “three or more”, etc. For example, at least one cell type means one, two, three, five, etc . . . cell types.

The term “about” particularly in reference to a given quantity, amount or number, is meant to encompass deviations of plus or minus ten (10) percent.

The phrase “alteration in the cell signature score” refers to a variation, either increase or decrease of said score when compared to a reference value or with the cell signature score determined previously. Preferably, this alteration or variation is statistically significant.

As used herein, the term “abundance” refers to a given quantity, amount, ratio or number of at least one cell type. This abundance is generally a relative abundance as it relates to a reference value. The abundance of at least one cell type can be expressed in units (e.g. cells/mm3) or as a percentage (%) of cells versus a reference standard, usually other cells.

In the last decade, several mathematical and machine learning methods have been developed to determine the relative abundance of a cell type in a biological mixture of different cell types, such as tumor tissue or blood, from genome-wide gene expression data. Some examples of these methods are EPIC, described by Racle et al 2017, CYBERSORT, described by Newman et al. Nature methods 2015; ImmuCellAI described by Miao et al 2020; xCell, described by Aran et al 2017. These methods, referred to as deconvolution methods, report accurate translation of gene expression levels into a relative quantification (proportion) of the different cell types in the mixture. These methods were validated by correlating the inferred cell abundance to direct quantification of the cell type of interest by flow cytometry.

As used herein the terms “subject”, or “patient” are well-recognized in the art, and, are used interchangeably herein to refer to a mammal, including dog, cat, rat, mouse, monkey, cow, horse, goat, sheep, pig, camel, and, most preferably, a human. In some aspects, the subject is a subject in need of treatment or a subject suffering from a disease or a subject that might be at risk of suffering from a disease. However, in other aspects, the subject can be a normal subject. The term does not denote a particular age or sex. Thus, adult and newborn subjects, whether male or female, are intended to be covered.

The present invention contemplates a method for determining if a biomarker signature correlates with a cell count of at least one cell type, the method comprising:

i) selecting at least one cell type and providing a biomarker signature for said cell type, said biomarker signature comprising at least one gene whose expression is associated with said cell type;

i) providing a test sample and computing a signature score corresponding to a level of expression of said gene of said biomarker signature in the test sample;

iii) determining a cell count score in the test sample representing the cell count of said at least one cell type;

iv) comparing the biomarker signature score and the cell count score to determine if the biomarker signature correlates with the cell count of said cell type.

Also disclosed is a method for detecting a disease in a subject by estimating the abundance of at least one cell type in a subject's test sample, the method comprising:

i) determining at least one cell type relevant for the detection of said disease;

ii) providing a biomarker signature for said cell type, said biomarker signature comprising at least one gene whose expression is associated with the abundance of said cell type;

iii) computing a cell signature score corresponding to a level of expression of said at least one gene in the biomarker signature in the test sample; and

iv) comparing the cell signature score with a reference value to deduce if the subject is suffering, or not, from said disease.

As used herein, a “cell type” refers to any cell found in the body of a subject. A cell type can be a cell from solid tissue or a circulating cell. For example, a cell type will be selected among the group comprising non-circulating or circulating cells, immune cells, circulating immune cells, and tumor cells, or a combination of one or more thereof.

A “sample” as used herein refers to a biological sample obtained from a healthy subject (control sample), a subject at risk (test sample), or suffering from a disease (disease sample).

Preferably, the sample is selected from the group comprising whole blood, a fractional component of whole blood, serum, serum exosomes, plasma, semen, saliva, tears, urine, fecal material, sweat, buccal smears, skin, and cancer cells, or a combination of one or more thereof. More preferably, the test sample is selected among the group comprising a blood sample, or a fractional component thereof, white blood cells, peripheral blood mononuclear cell (PBMC), tumor sample, saliva, urine and other bodily fluids, or a combination of one or more thereof.

A “biomarker signature” or “cell type specific signature” refers to a set of genes and, in particular, to a set of gene expression products (proteins, metabolites and/or transcripts) that are associated with a specific cell type and/or a disease. In a preferred aspect, the biomarker signature comprises a set of at least one gene, preferably between 2-500 genes, more preferably between 10-300 genes, most preferably between 20-250 genes, even more preferably between 3-25 genes, whose expression is associated with said cell type.

The “at least one gene” refers to any gene which expression is found in the body of a subject and associated with a specific cell type.

Non-limiting examples of genes composing the signatures are selected among those listed in the following tables, or among a (sub)set of the genes listed in the following tables:

TABLE 1
Gene list for the T cell-specific signature

| Gene ID * | Gene symbol | Gene description |
|---|---|---|
| ENSG00000065357 | DGKA | diacylglycerol kinase alpha |
| ENSG00000071575 | TRIB2 | tribbles pseudokinase 2 |
| ENSG00000081059 | TCF7 | transcription factor 7 |
| ENSG00000100100 | PIK3IP1 | phosphoinositide-3-kinase interacting protein 1 |
| ENSG00000101842 | VSIG1 | V-set and immunoglobulin domain containing 1 |
| ENSG00000103351 | CLUAP1 | clusterin associated protein 1 |
| ENSG00000104660 | LEPROTL1 | leptin receptor overlapping transcript like 1 |
| ENSG00000115687 | PASK | PAS domain containing serine/threonine kinase |
| ENSG00000117602 | RCAN3 | RCAN family member 3 |
| ENSG00000126353 | CCR7 | C-C motif chemokine receptor 7 |
| ENSG00000135426 | TESPA1 | thymocyte expressed, positive selection associated 1 |
| ENSG00000136111 | TBC1D4 | TBC1 domain family member 4 Symbol |
| ENSG00000138795 | LEF1 | lymphoid enhancer binding factor 1 |
| ENSG00000140511 | HAPLN3 | hyaluronan and proteoglycan link protein 3 |
| ENSG00000140743 | CDR2 | cerebellar degeneration related protein 2 |
| ENSG00000147457 | CHMP7 | charged multivesicular body protein 7 |
| ENSG00000152495 | CAMK4 | calcium/calmodulin dependent protein kinase IV |
| ENSG00000154153 | RETREG1 | reticulophagy regulator 1 |
| ENSG00000154229 | PRKCA | protein kinase C alpha |
| ENSG00000154814 | OXNAD1 | oxidoreductase NAD binding domain containing 1 |
| ENSG00000164530 | PI16 | peptidase inhibitor 16 |
| ENSG00000166313 | APBB1 | amyloid beta precursor protein binding family B member 1 |
| ENSG00000167106 | FAM102A | family with sequence similarity 102 member A |
| ENSG00000171843 | MLLT3 | MLLT3 super elongation complex subunit |
| ENSG00000172005 | MAL | T cell differentiation protein |
| ENSG00000184613 | NELL2 | neural EGFL like 2 |

TABLE 2
Gene list for the B cell-specific signature

| Gene ID* | Gene symbol | Gene description |
|---|---|---|
| ENSG00000077238 | IL4R | interleukin 4 receptor |
| ENSG00000100721 | TCL1A | T cell leukemia/lymphoma 1A |
| ENSG00000104921 | FCER2 | Fc fragment of IgE receptor II |

TABLE 3
Gene list for the NK cell-specific signature

| Gene ID* | Gene symbol | Gene description |
|---|---|---|
| ENSG00000021762 | OSBPL5 | oxysterol binding protein like 5 |
| ENSG00000101082 | SLA2 | Src like adaptor 2 |
| ENSG00000108370 | RGS9 | regulator of G protein signaling 9 |
| ENSG00000109943 | CRTAM | cytotoxic and regulatory T cell molecule |
| ENSG00000115607 | IL18RAP | interleukin 18 receptor accessory protein |
| ENSG00000139116 | KIF21A | kinesin family member 21A |
| ENSG00000149294 | NCAM1 | neural cell adhesion molecule 1 |
| ENSG00000156475 | PPP2R2B | protein phosphatase 2 regulatory subunit B beta |
| ENSG00000171916 | LGALS9C | galectin 9C |

TABLE 4
Gene list for the monocyte-specific signature

| Gene ID* | Gene symbol | Gene description |
|---|---|---|
| ENSG00000105383 | CD33 | CD33 molecule |
| ENSG00000106066 | CPVL | carboxypeptidase vitellogenic like |
| ENSG00000121807 | CCR2 | C-C motif chemokine receptor 2 |
| ENSG00000138744 | NAAA | N-acylethanolamine acid amidase |
| ENSG00000155465 | SLC7A7 | solute carrier family 7 member 7 |
| ENSG00000158473 | CD1D | CD1d molecule |
| ENSG00000165168 | CYBB | cytochrome b-245 beta chain |

TABLE 5
Gene list for the neutrophil-specific signature

| Gene ID* | Gene symbol | Gene description |
|---|---|---|
| ENSG00000011198 | ABHD5 | abhydrolase domain containing 5, lysophosphatidic acid |
| | | acyltransferase |
| ENSG00000059728 | MXD1 | MAX dimerization protein 1 |
| ENSG00000059804 | SLC2A3 | solute carrier family 2 member 3 |
| ENSG00000087903 | RFX2 | regulatory factor X2 |
| ENSG00000093134 | VNN3 | vanin 3 |
| ENSG00000105835 | NAMPT | nicotinamide phosphoribosyltransferase |
| ENSG00000112096 | SOD2 | superoxide dismutase 2 |
| ENSG00000124731 | TREM1 | triggering receptor expressed on myeloid cells 1 |
| ENSG00000129657 | SEC14L1 | SEC14 like lipid binding 1 |
| ENSG00000161921 | CXCL16 | C—X—C motif chemokine ligand 16 Symbol |
| ENSG00000173334 | TRIB1 | tribbles pseudokinase 1 |
| ENSG00000186431 | FCAR | Fc fragment of IgA receptor |
| ENSG00000187116 | LILRA5 | leukocyte immunoglobulin like receptor A5 |
| ENSG00000197852 | INKA2 | inka box actin regulator 2 |

*Human Protein Atlas (Uhlen et al Science 2019 , http://www.proteinatlas.org)

The expression of a gene can be detected and/or measured, directly or indirectly, from a nucleic acid or a protein, or a combination thereof. Examples of nucleic acids from which the gene expression can be detected and/or measured comprise deoxyribonucleotide (e.g. DNA, cDNA, . . . ) or ribonucleotide (e.g. RNA, mRNA, miRNA, siRNA, piRNA, hnRNA, snRNA, esiRNA, shRNA, lncRNA, . . . ). Preferably, the nucleic acid is a deoxyribonucleotide, most preferably an mRNA.

The level of an RNA, preferably an mRNA, in a biological sample can be measured or determined using any technique that is suitable for detecting RNA expression levels in a biological sample. Suitable techniques for determining RNA, preferably an mRNA, expression levels in cells from a biological sample (e.g. Northern blot analysis, RT-PCR, quantitative RT-PCR, microarray, in situ hybridization, serial analysis of gene expression (SAGE), immunoassay, mass spectrometry, and any sequencing-based methods known in the art such as RNA-seq or Next-generation sequencing) in the methods of the invention are well known to those of skill in the art.

Alternatively, the level of an RNA, preferably an mRNA, in a biological sample can be detected, measured and/or determined indirectly by measuring abundance levels of cDNAs, amplified RNAs or DNAs, or by measuring quantities or activities of RNAs, or other molecules that are indicative of the expression level of the RNA. Preferably, the level of an RNA, e.g. an mRNA, in a biological sample is determined indirectly in the methods of the invention by measuring abundance levels of cDNAs.

Preferably, the computing step is performed by a computation tool selected from the group comprising an automated computation tool selected from the group comprising at least one mathematical formula, at least one computational step, and at least one algorithm, or a combination thereof.

In an aspect of the invention, the reference value is the median expression of the genes composing the signature in at least one healthy patient. Alternatively, the reference value is the median expression of the genes composing the signature in at least one patient suffering from a disease.

In some aspects of the present invention, the reference value is the expression level of a particular biomarker signature of interest, such as the biomarker signature score, in a sample obtained from the same subject prior to any disease treatment (e.g. cancer). In other aspects of the present invention, the reference value is the expression level of a particular biomarker of interest in a sample obtained from the same subject during a treatment and not responsive to said treatment. Alternatively, the reference value is a prior measurement of the expression level of a particular gene of interest in a previously obtained sample from the same subject or from a subject having similar age range, disease status (e.g., stage) to the tested subject.

The reference value is usually determined from a patient or set of patients of a similar race, ethnicity, sex, demographic and/or genetic background, or a combination thereof as the patient providing the test sample.

Such reference values can be derived from statistical analyses and/or risk prediction data of populations obtained from mathematical algorithms. Reference indices can also be constructed by the person skilled in the art and used utilizing algorithms and other methods of statistical and structural classification.

In an aspect of invention, the method for determining if a biomarker signature correlates with a cell count of at least one cell type consists in a procedure of combining, e.g. publicly available knowledge with a data driven approach to identify gene expression signature highly specific for a cell type (cell tissue or a circulating cell).

A repertoire of candidate genes for, e.g. the transcriptomic signature related to the cell type is constructed from the merge of previously published consensus signatures and public databases.

The candidate genes repertoire is then filtered out for lowly expressed genes by comparing the expression levels in the organ of interest, setting a threshold to preferably about 3 transcripts per million (TPM), more preferably about 5 transcripts per million (TPM), even more preferably 5 transcripts per million (TPM) to retain the reliably measurable genes.

Gene correlation analysis of the entire gene repertoire is performed on at least three public and/or private datasets to identify highly correlated gene clusters among the selected biomarkers, in each dataset.

Gene clusters of each dataset are analyzed by functional analysis and the best candidate cluster per dataset is identified based on its specificity to the cell type.

Each dataset best candidate gene clusters is refined to a core gene signature, composed of the overlapping genes among all dataset's best cluster.

Finally, the gene signature specificity for the biological target is validated on an independent transcriptomic dataset derived from the purified or enriched target cell type.

The present invention allows determination of the correlation between cell counts and biomarker signatures and evaluation of the potential of these signatures for, for example, a disease detection.

The inventors have shown that biomarker signature scores of specific immune cell types correlate with traditional cell counting methods, enabling the extraction of valuable clinical information from transcriptomic data.

Advantageously, the present invention provides high-performance convenient test, in particular from body liquid such as blood, for early cancer detection.

The biomarker signature score may be calculated as the mean, or the median or the sum of the expression levels of the genes composing the signature in control samples and disease samples. Alternatively, the score may be calculated as the first component or multiple components of principal component analysis (PCA), or as low dimensional embeddings using neural networks.

As used herein a disease refers to any abnormal condition that negatively affects the structure or function of all or part of an organism. In an aspect of the invention, the disease is selected among the non-limiting group comprising an infection disease (due to a virus or a bacteria), an immunological disease, cancer and hematological disorders. Preferably, the disease is cancer or infection disease. Most preferably, the disease is advance adenoma (AA), colorectal cancer (CRC), bladder cancer or tuberculosis.

In an aspect of the invention, the cell count score in the test sample is determined by hematology testing, or a manual system such as counting chamber, or by immunohistochemistry, or an automated system such as a flow cytometry device, or a combination thereof.

In an aspect of the invention, a cell signature score superior to the reference value indicates that the test sample is positive for the disease, and a cell signature score inferior to the reference value indicates that the test sample is negative for the disease. As shown in the examples, monocyte and neutrophil cell signature scores significantly increases in CRC subjects.

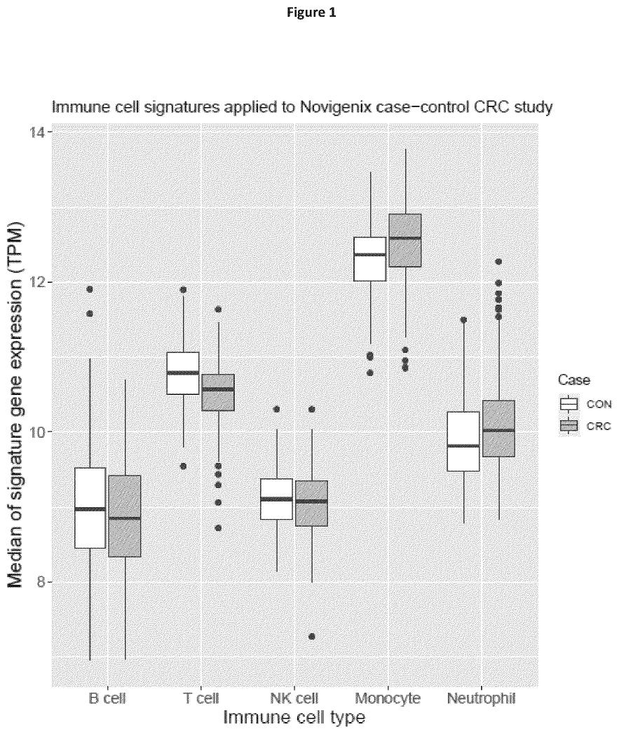

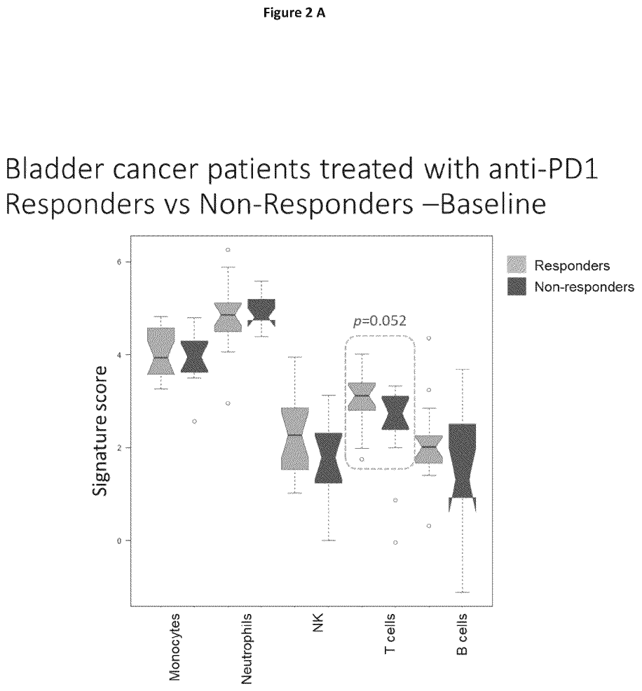

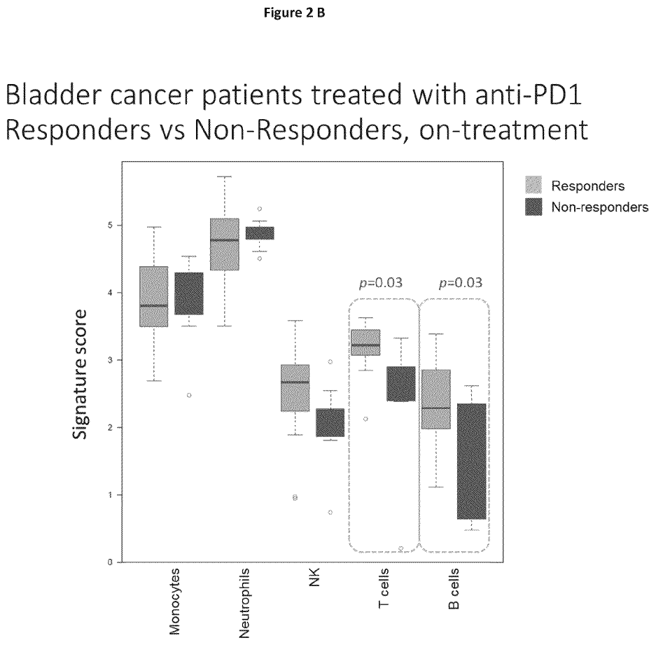

Alternatively, in certain aspects of the invention, a cell signature score superior to the reference value indicates that the test sample is negative for the disease, and a cell signature score inferior to the reference value indicates that the test sample is positive for the disease. This is the case, e.g., for the T cell signature score that shows significant decrease in CRC patients (FIG. 1).

Furthermore, the discriminatory power of the signatures can be enhanced when the cell type signature score is a ratio of cell type signature scores such as, e.g., the ratio of neutrophils/T cells or monocytes/T cells.

This indicate that the neutrophil, monocyte and T cell signature scores can be used as biomarker for cancer detection, particularly for the detection of CRC.

The present invention further relates to a method for determining the progression or regression of a disease in a subject suffering therefrom, said method comprising:

i) computing a cell signature score corresponding to a level of expression of at least one gene in the biomarker signature in a test sample obtained form said subject; and

ii) periodically comparing the cell signature score with a reference value or with the cell signature score determined previously,

wherein an alteration in the cell signature score associated with the abundance of at least one cell type in said biological sample, relative to the reference value or the cell signature score determined previously, is indicative of the progression or regression of said disease.

Further provided herein is a method of stratifying a disease in a subject suffering therefrom, said method comprising:

i) providing a biomarker signature for a cell type relevant for the detection of said disease, said biomarker signature comprising at least one gene whose expression is associated with the abundance of said cell type;

ii) computing a cell signature score corresponding to a level of expression of said at least one gene in the biomarker signature in the test sample; and

iii) comparing the cell signature score with a reference value,

wherein a cell signature score superior or inferior to the reference value is indicative of the disease stage or grade.

Also provided is a method for determining if a subject suffering from a disease is responsive to a treatment, said method comprising

i) computing a cell signature score corresponding to a level of expression of at least one gene in the biomarker signature in a test sample obtained from said subject, and

ii) periodically comparing the cell signature score with a reference value or with the cell signature score determined previously,

wherein an alteration in the cell signature score associated with the abundance of at least one cell type in said biological sample, relative to the reference value or the cell signature score determined previously, is indicative of the responsiveness of the subject to the treatment.

In case the disease is cancer, then the treatment is preferably selected from the group comprising surgery, radiotherapy, chemotherapy, immunotherapy or hormone therapy. Examples of the immunotherapy include T-cell transfer therapy, monoclonal antibodies, vaccines, and immune system modulators such as e.g. immune checkpoint inhibitors.

Examples of immune checkpoint inhibitor are selected from the group comprising PD-1 inhibitor (e.g. Nivolumab, Pembrolizumab, . . . ), PD-L1 inhibitor, and CTLA-4 inhibitor, or a combination thereof.

Examples of chemotherapy are selected from the group of drugs comprising doxorubicin, carboplatin, cyclophosphamide, epirubicin, fluorouracil (5-FU), methotrexate, paclitaxel, docetaxel, or a combination of one or more of these drugs.

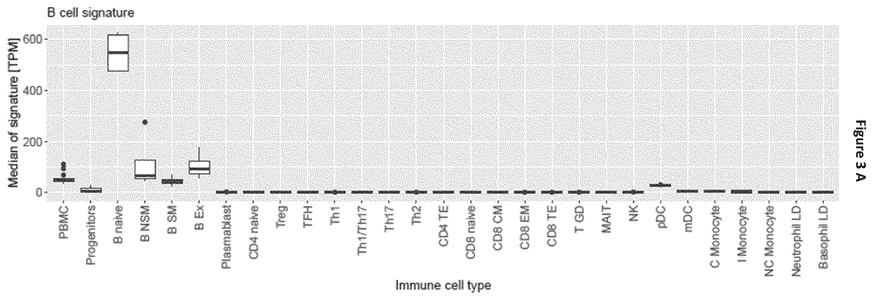

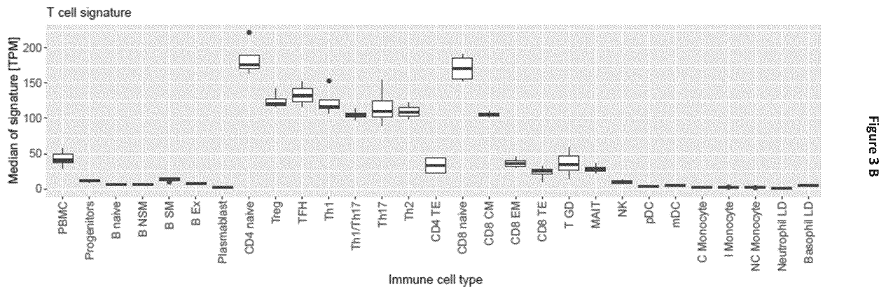

Referring in more details to Example 2, analysis of the immune gene signature at baseline shows that there is T cells enrichment in the blood of responders compared to non-responders (FIG. 2A).

During treatment, the enrichment of the T cells was shown to be even bigger in the responders and at this time point B cells also appeared to be enriched. This is in line with the expected T cells and adaptive response activation due to the response to the anti-PD 1 treatment (FIG. 2).

In an aspect of the invention, the methods described herein further comprise a step of administering a pharmaceutical composition for treating the disease or adapting the treatment by modifying the regimen, the mode of administration and/or the pharmaceutical composition.

In an aspect of the invention, the methods described herein are computer-implemented methods.

Also contemplated is a kit for performing a method according to the invention, said kit comprising a) means and/or reagents for determining the expression level of one or more gene whose expression is associated with the abundance of a cell type in a test sample obtained from a subject, and b) instructions for use. Preferably, the means consist in an assay, preferably an RNA-seq on the Illumina platform.

For example, the kit may include reagents that specifically hybridize to one or more gene or gene expression product of the invention. Such reagents may be one or more nucleic acid molecule in a form suitable for detecting the expression of the one or more gene of the invention, for example, a probe or a primer. The kit may include reagents useful for performing an assay to detect the expression of the one or more gene of the invention, for example, reagents which may be used to detect one or more gene transcripts in a RT-PCR reaction. The kit may likewise include a microarray useful for detecting one or more gene of the invention.

Probes and/or primers can be selected from those provided in the scientific literature or specifically designed for detecting the expression of the one or more gene of the invention.

The kit may further contain instructions for suitable operational parameters in the form of a label or product insert. For example, the instructions may include information or directions regarding how to collect a sample, how to determine the level expression of the one or more gene of the invention, or how to correlate the level of expression of the one or more gene of the invention in a sample with the status of a subject.

Also provided herein is a device for performing a method of the invention, said device comprising:

i) a sample chamber for a test sample collected from a subject;

ii) an assay module in fluid communication with said sample chamber, said assay module comprising means and/or reagents for detecting and/or measuring, directly or indirectly, the gene expression in said test sample;

iii) means for computing a cell signature score; and

iv) a user interface wherein said user interface relates the cell signature score to detecting a disease in said subject, stratifying a disease or determining the responsiveness to a treatment.

Also provided is a method to identify at least one gene expression signature highly specific for a given cell type, the method comprising:
i. compiling a repertoire of candidate genes for said cell type from, e.g., previously published consensus signatures and/or public databases,
ii. filtering the candidate gene repertoire for lowly expressed and highly variable genes by comparing the expression levels in the organ of interest, setting a threshold to retain the reliably measurable genes,
iii. clustering the genes based on their correlation on at least three public and/or private datasets and selecting highly correlated gene clusters, in each dataset,
iv. confirming the specificity of the selected gene clusters of each dataset by functional analysis,
v. identifying a core gene signature defined as the gene overlap among the gene clusters selected in each dataset, and
vi. validating the specificity of the gene signature for the target cell type on an independent gene expression dataset derived from the purified or enriched target cell type.

Preferably, the gene signature consists in a transcriptomic signature and the threshold corresponds to about 5 transcripts per million (TPM).

Also provided herein is the use of at least one gene of a cell specific signature selected from the group comprising, or consisting of, the genes of Table 1, Table 2, Table 3, Table 4 and/or Table 5 for in methods for detecting a disease.

As disclosed herein, the methods of the present invention allow an estimate cell count or abundance in an advantageous manner to overcome the drawbacks of the existing methods. The cell abundance estimation or cell count is determined by studying the expression of the gene(s) composing the biomarker signature.

The present invention allows definition of cell types specific signatures based on the expression profiles of genes, for instance mRNA sequences.

The present invention allows comparison of the cell type signatures to the standard cell counting testing for each sample/subject.

The present invention allows analyzing how the expression profiles of a biomarker signature, for instance mRNA sequencing data, in the different cell type signatures differ depending on the samples, for instance between a control (no disease), advance adenoma (AA), colorectal cancer (CRC), and other disease for instance other cancers (OC).

The present invention allows analyzing how the expression profiles of mRNA sequencing data in the different cell type signatures differ in two populations, such as an Asian and a Caucasian population.

Further particular advantages and features of the invention will become more apparent from the following non-limitative description the examples of at least one embodiment of the invention which will refer to the accompanying drawings.

The present detailed description is intended to illustrate the invention in a non-limitative manner since any feature of an embodiment may be combined with any other feature of a different embodiment in an advantageous manner.

### EXAMPLES

### Example 1

The Use of Immune Cell Signatures for Cancer Detection

Methods: The transcriptome profiles of peripheral blood mononuclear cells (PBMC) from 561 Asian and Caucasian subjects, including 189 CRC, 115 advanced adenomas, 39 other cancers, 218 controls without any colorectal lesions (CON) were generated by RNA-seq on the Illumina platform. Subjects were older than 50 years, referred to a screening or diagnostic colonoscopy or scheduled for CRC resection.

Neutrophils, lymphocytes and monocytes counts were obtained by standard hematology testing, such as complete blood count with differentials. Immune cell gene signatures, specific to T cells, B cells, NK cells, monocytes and neutrophils were generated as explained in example 1.

Sequencing libraries were prepared using the TruSeq Stranded mRNA Library Prep kit (Illumina) with polyA selection. Paired-end sequencing was performed on the Illumina HiSeq 4000 platform, with a depth of 30M reads/sample. For each sample, gene transcripts were quantified as transcript per million (TPM) using Salmon analytical pipeline.

For each subject the gene expression median of each cell type signature gene set has been calculated to measure a subject's cell type signature. The results with the median were promising, robust and better correlated to reference cell counts, therefore the median of each cell type gene set signature was selected as a subject's cell type signature measure.

Cell signatures based on gene RNAseq expression median values are compared between healthy control (CON) subjects and patients with colorectal cancer (FIG. 1). Monocyte and Neutrophil cell signature score significantly increases in CRC subjects. In contrast, T cell signature score shows significant decrease in CRC patients (FIG. 1). Mann-Whitney U-test—analysis has been performed and P-value results show that monocyte cell signature was the most significant (table 6). The discriminatory power of the signatures is even bigger by calculating the ratios between Neutrophil/T cells and Monocyte/T cells. This indicate that the neutrophil, monocyte and T cell signature score can be used as biomarker for cancer detection.

TABLE 6
Summary of comparison of cell signatures
between CRC and CON groups. Results of the
Mann-Whitney U-test are displayed as p-values.

| | signature score | |
|---|---|---|
| | variation in | |
| | CRC vs CON | P-value |
| Cell type | | |
| Neutrophils | increase | 2.2 × 10⁻³ |
| Monocytes | increase | 6.1 × 10⁻⁷ |
| T cells | decrease | 9.5 × 10⁻⁹ |
| NK cells | equal | 0.33 |
| B cells | equal | 0.16 |
| Cell type ratio | | |
| Neutrophils/T cells | increase | 2.14 × 10⁻⁷ |
| Monocytes/T cells | increase | 2.35 × 10⁻¹⁰ |

As a confirmation that the discriminative potential of the monocyte, neutrophil and T cell signature is due to a variation of the cell number in blood, we compared monocyte, neutrophil and lymphocyte blood counts in cancer group and the healthy control group. The results were similar to the one obtained with the cell gene expression signatures. Student's t-test analysis has been performed and the P-value results show that neutrophil (p-values=6.12×10⁻¹¹) and Monocyte (p-values=6.8×10⁻⁶) count is significantly increased in the CRC group compared to the CON group. On the contrary, the lymphocyte count shows a tendency to decrease in CRC compared to CON group, but not reaching statistical significance. The median of the immune cell signature, or the sum of medians, is correlated with the immune cell counts of the 571 matched samples data. The correlation coefficient estimate is calculated from the fitting of a linear model to the two correlated parameters.

This demonstrate that the gene signature score is reliable parameter to estimate a relative cell abundancy.

This study shows that measuring specific immune cell type by RNA signatures correlate with traditional cell counting methods, enabling the extraction of valuable clinical information from blood transcriptomic data. This data suggests that blood myeloid and T cells measured by RNA signatures are promising biomarkers for CRC detection.

An association between cell count and patient disease status was observed. An immuno-transcriptomic cell signature was validated and correlates with traditional cell count measurements. Cell signature is thus a potential biomarker for CRC detection. The non-invasive character of the blood transcriptomic approach makes it a potential alternative for CRC screening.

The present example demonstrates that:
Neutrophil and monocyte gene signature are positively associated with the presence of CRC;
T cell gene signature is negatively associated with the presence of CRC;
The neutrophil-to-T cell and monocyte-to-T cell signature ratios increased the discrimination power of CRC compared to CON group
Immune cell type signature generally correlates with cell counts

### Example 2

Use of Immune Cell Signatures to Predict Cancer Treatment Response and Monitor Treatment

A single-center, retrospective study was conducted in 31 consecutive patients with metastatic Urothelial Cancer (UC) treated with anti-PD-1. Whole blood samples were collected in PAXgene Blood RNA tubes before (baseline) and after 2-6 weeks 8 on-treatment) of anti-PD-1 therapy. Clinical benefit was defined as progression-free survival (PFS)≥6 months. In total, 18 patients experienced clinical benefit (CB+) and 13 did not (CB−) (Table 7).

TABLE 8
Patient characteristics

| | |
|---|---|
| Male - n (%) | 24 (77.4) |
| Age - median (range) | 68 (38-80) |
| Treatment - n (%) | |
| Nivolumab | 8 (25.8) |
| Pembrolizumab | 23 (74.2) |
| Previous platinum-based chemotherapy - n (%) | 27 (87.1) |
| Location of metastases - n (%) | |
| Lymph node only | 9 (29.0) |
| Visceral metastases | 17 (54.8) |
| Liver metastases | 7 (22.6) |
| Clinical outcome - n (%) | |
| PFS < 6 months | 13 (41.9) |
| PFS ≥ 6 months | 18 (58.1) |
| Compete response* | 5 (16.1) |
| Partial response* | 10 (32.3) |
| Stable disease* | 2 (6.5) |
| not evaluable* | 1 (3.2) |

*Objective response according to RECIST1.1

Patients without clinical benefit (CB−) have been classified as non-responders, and patients with clinical benefit (CB+) as responders.

Analysis of the immune gene signature at baseline indicate that there is T cells enrichment in the blood of responders compared to non-responders (as shown in FIG. 2).

During treatment, the enrichment of the T cells was shown to be even bigger in the responders and at this time point B cells also appeared to be enriched. This is in line with the expected T cells and adaptive response activation due to the response to the anti-PD 1 treatment (as shown in FIG. 2).

### Example 3

Selection of Gene Signatures Specific to T Cells, B Cells, NK Cells, Monocytes and Neutrophil

Immune cell gene signatures, specific to T cells, B cells, NK, monocytes and neutrophils were generated based on the method described in example 1.

The repertoire of candidate genes were defined by using recently published signatures (Racle et al 2017, Palmer et al 2006, Newman et al 2015, Miao et al 2020, Aran et al 2017) and by using the blood dataset of Human Protein Atlas (Uhlen et al Science 2019, http://www.proteinatlas.org). The Blood Atlas contains single cell type information on genome-wide RNA expression profiles of human protein-coding genes covering various B- and T-cells, monocytes, granulocytes and dendritic cells. The single cell transcriptomics analysis covers 18 cell types isolated with cell sorting followed by RNA-seq analysis. Candidate genes were extracted from the cell lineage enriched genes specific to each blood cell type from the Blood atlas.

For the 5 immune cell types analysed, we identified a repertoire of candidate genes varying between 338-1392 genes.

Gene expression values of the candidate genes were calculated in an unpublished RNA seq dataset generated from peripheral blood mononuclear cells (PBMC) and low expressed genes (<5 TPM) were filtered out. Further filtering was applied by identifying the most correlated genes within each signature. The correlation analysis was performed independently on 3 unpublished RNAseq datasets, 2 generated from 561 PBMC samples of healthy donors and colorectal cancers patients (described in Example 1) and one from 59 whole blood samples of metastatic bladder cancer patients treated with anti-PD-1.

The best correlation clusters were confirmed through functional and network analysis performed with the webtools EnrichR (Chen et al. BMC Bioinformatics 2013, Kuleshov et al. Nucleic Acid Research 2016) and STRING (Snel et al. Nucleic Acid Research 2000, Szklarczyk et al Nucleic Acid Research 2019) respectively.

A final consensus gene list for each cell signature was determined by identifying the overlapping genes identified in the correlation analysis on the 3 datasets. The genes of each signatures are listed in tables 1-5.

The specificity of the cell signatures was tested on the Monaco's RNAseq dataset (Monaco et al. Cell Reports 2019). These data are available from GEO: GSE107011. This RNAseq dataset includes PBMC data of 13 Singaporean blood donors, as well as data from 28 different immune cell types purified by flow cytometry, in 4 replicates, except for T CD4 TE (2 replicates) and T GD (8 replicates).

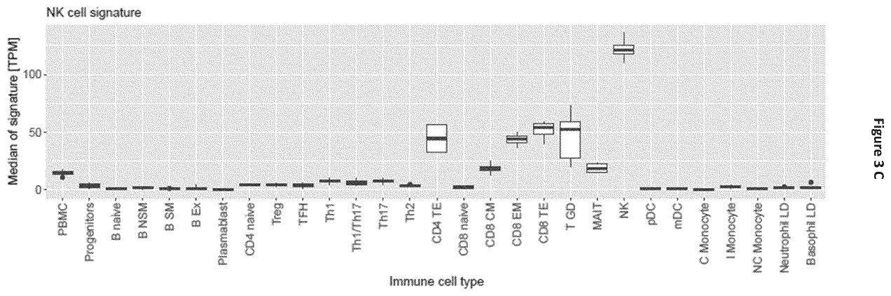

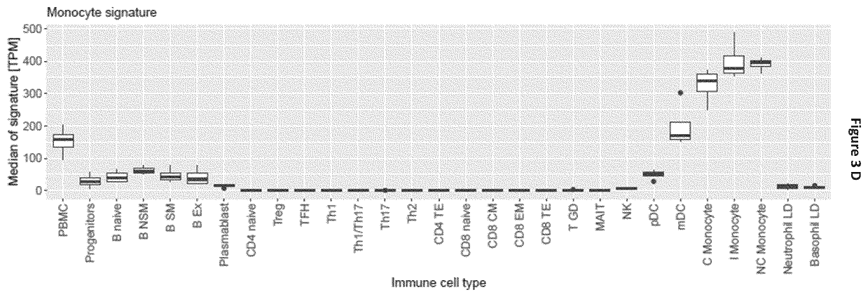

To this end a cell signature score was calculated as the median of the expression values (TPM) of all the genes within a given signature in one sample and the signature score compared across the 28 different cell types of Monaco's dataset. As illustrated in FIG. 3, all the identified signature scores are significantly expressed only in the immune cell types related to the signature of interest.

For instance, the monocyte signature shows significant expression only in the monocyte related cell types, i.e. monocytic dendritic cells (mDC), Classic, Intermediate and Non-Classic monocytes, and PBMC.

### Example 4

The Use of the Immune Cell Signature Score to Estimate Relative Blood Immune Cell Abundance.

According to WHO, Tuberculosis is in the top 10 of mortality causes worldwide (https://www.who.int) and one of the first cause of mortality in HIV patients. In 2019, WHO estimated that 10 Mio persons were newly infected with TB. This infectious disease is caused by the bacterium Mycobacterium tuberculosis, an airborne pathogen, which most of the time infects the patient's lungs and can either remain latent or develop, especially in immunodeficient or smoking patients. Treatment of TB involved antibiotics drugs cocktails for 4 to 6 months, until the patient is declared TB-free. In the case of multiresistant TB, the treatment time is extended, and mortality rate increased.

To further validate the ability of the identified immune cell signatures to detect disease cases compared to healthy controls based on immune cell signature score, we searched for an independent public RNA-Seq data of case-control study, where changes in the immune cell blood proportion were documented. We selected a Tuberculosis treatment study, for its high sample size and the availability of samples without treatment for both the cases and the healthy controls.

Public RNA-Seq data were retrieved from the GEO public repository (https://www.ncbi.nlm.nih.gov/geo), under the accession number GSE89403. The study consists in RNA-Seq data generated from a total of 914 whole blood samples (PAXgene), including 100 TB cases and 38 healthy controls from South Africa (Cape Town) enrolled in a longitudinal monitoring during TB treatment between 2010 and 2013 (Thompson et al. Tuberculosis 2017). All the patients were tested negative to HIV at the enrollment time. Only the samples withdrawn at baseline (prior any treatment) were used in this analysis, which consisted in 91 TB cases and 24 healthy controls, each measured in duplicates.

RNAseq data were filtered out for lowly expressed genes and then normalized (VST) according to standard RNA-Seq data treatment. The median of each immune cell signature is calculated on the baseline samples for both the healthy controls and TB cases.

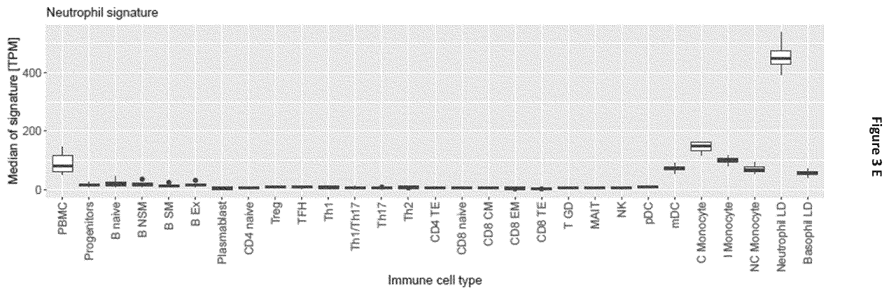

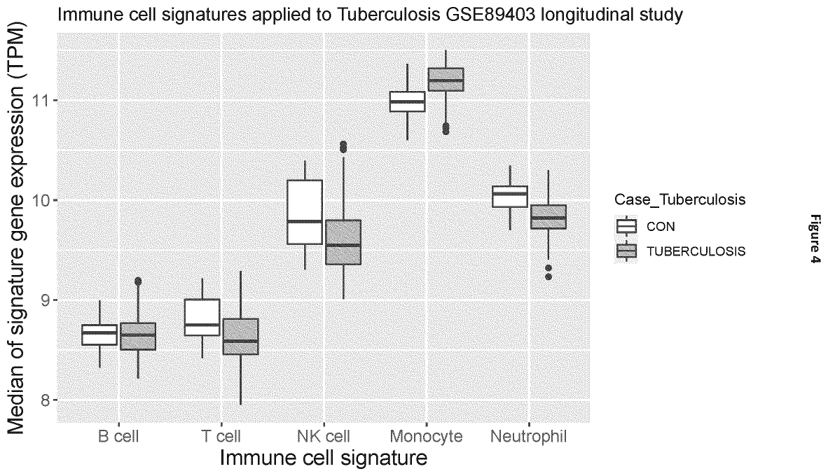

As shown in FIG. 4, the monocyte signature score, calculated as the median of gene signature, shows indeed a significantly higher expression level in the TB cases than in the healthy controls.

However, this innate response is sometimes not sufficient to get rid of TB infection, with bacteria infecting their monocytic host. Natural Killer (NK) cells have been shown to be essential to the activation and regulation of the adaptive response in TB patients. Indeed, through interferon gamma (IFN-gamma) secretion, they promote CD8+T cell proliferation and effector function against host TB-infected phagocytic cells (Vankayalapati et al. The Journal of Immunology 2004). Thus, NK cells and T cells blood depletions are associated with TB-infected patients (Cai et al. The lancet 2020, Rodrigues et al. Clinical and Experimental Immunology 2002). FIG. 4 shows indeed a decrease of NK and T cell signature score in the TB cases compared to the controls, recapitulating what observed using traditional cell count methods.

These data confirm that the immune cell signature score are specific to the immune cell type of interest and that can be used in substitution of traditional methods for blood immune cells abundance estimation.

TABLE 9
summary of the statistics performed on the immune cell
signature score on TB versus healthy controls (CON).

| TB vs | | | | | |
|---|---|---|---|---|---|
| CON | B cell | T cell | NKcell | Monocyte | Neutrophils |
| P-value | 0.4817 | 9.33e−06 | 7.19e−06 | 4.39e−11 | 9.41e−13 |
| Balance | equal | decreased in | decreased in | increased in | decreased in |
| | | TB | TB | TB | TB |

Significance is assessed with a two-sample non-paired Wilcoxon test, also known as Mann-Whitney test, with a 95% confidence level. Balance indicates the relative levels of TB and CON medians.

While the embodiments have been described in conjunction with several embodiments, it is evident that many alternatives, modifications and variations would be or are apparent to those of ordinary skill in the applicable arts. Accordingly, this disclosure is intended to embrace all such alternatives, modifications, equivalents and variations that are within the scope of this disclosure. This for example particularly the case regarding the different apparatuses which can be used.

1. A method for detecting a disease in a subject by estimating the relative abundance of at least one cell type in a subject's test sample, the method comprising:
i) determining at least one cell type relevant for the detection of said disease;
ii) providing a biomarker signature for said cell type, said biomarker signature comprising at least one gene whose expression is associated with the abundance of said cell type;
iii) computing a cell signature score corresponding to a level of expression of said at least one gene in the biomarker signature in the test sample; and
iv) comparing the cell signature score with a reference value to deduce if the subject is suffering, or not, from said disease.

2. The method according to claim 1, wherein the cell type is selected from the group consisting of non-circulating cells, circulating cells and a combination thereof.

3. The method according to claim 1, wherein the disease is selected from the group consisting of a cancer, an infectious diseases, an immune diseases and a hematological disorders.

4. The method according to claim 1, wherein
i) a cell signature score superior to the reference value indicates that the test sample is positive for the disease, or
ii) a cell signature score inferior to the reference value indicates that the test sample is negative for the disease.

5. The method according to claim 1, wherein the cell type is selected from the group consisting of neutrophils and monocytes and a combination thereof.

6. The method according to claim 1, wherein
i) a cell signature score inferior to the reference value indicates that the test sample is positive for the disease, or
ii) a cell signature score superior to the reference value indicates that the test sample is negative for the disease.

7. The method according to claim 1, wherein the cell type is selected from the group consisting of T cells and NK cells and a combination thereof.

8. The method according to claim 1, wherein the computing step is performed by a computation tool selected from the group consisting of an automated computation tool selected from the group consisting of at least one mathematical formula, at least one computational step, at least one algorithm and a combination thereof.

9. The method according to claim 1, wherein said gene expression is detected and/or measured, directly or indirectly, from a nucleic acid or a protein, or a combination thereof.

10. The method according to claim 1, wherein the cell type is circulating immune cells, the disease is cancer, preferably a colorectal cancer, and the gene biomarker signature is detected and/or measured, directly or indirectly, from a nucleic acid, preferably RNA.

11. The method according to claim 1, wherein the reference value is the mean expression of the genes composing the signature in at least one healthy patient.

12. The method according to claim 1, wherein the reference value is the mean expression of the genes composing the signature in i) at least one patient suffering from a disease or ii) in at least one healthy patient.

13. The method according to claim 1 wherein the sample is selected from the group consisting of a blood sample or a fractional component thereof, white blood cells, PBMC and a combination thereof.

14. The method according to claim 1, wherein the disease is Colorectal Cancer (CRC).

15. The method according to claim 1, wherein the cell type is circulating immune cells selected from the group consisting of neutrophils, monocytes, T cells, B cells, NK cells and a combination thereof.

16. The method according to claim 1, wherein the reference values are determined from a patient or set of patients of a similar race, ethnicity, sex, demographic and/or genetic background, or a combination thereof as the patient providing the test sample.

17. The method according to claim 1, wherein the cell type signature score is a ratio of cell type signature scores.

18. The method according to claim 17, wherein the ratio of cell types is Neutrophils/T cells or monocytes/T cells.

19. A method for determining the progression or regression of a disease in a subject suffering therefrom, said method comprising:
i) computing a cell signature score corresponding to a level of expression of at least one gene in the biomarker signature in a test sample obtained form said subject; and
ii) periodically comparing the cell signature score with a reference value or with the cell signature score determined previously,
wherein an alteration in the cell signature score associated with the abundance of at least one cell type in said biological sample, relative to the reference value or the cell signature score determined previously, is indicative of the progression or regression of said disease.

20. A method of stratifying a disease in a subject suffering therefrom, said method comprising:
i) providing a biomarker signature for a cell type relevant for the detection of said disease, said biomarker signature comprising at least one gene whose expression is associated with the abundance of said cell type;
ii) computing a cell signature score corresponding to a level of expression of said at least one gene in the biomarker signature in the test sample; and
iii) comparing the cell signature score with a reference value,
wherein a cell signature score superior or inferior to the reference value is indicative of the disease stage or grade.

21. A method for determining if a subject suffering from a disease is responsive to a treatment, said method comprising
i) computing a cell signature score corresponding to a level of expression of at least one gene in the biomarker signature in a test sample obtained from said subject, and
ii) periodically comparing the cell signature score with a reference value or with the cell signature score determined previously,
wherein an alteration in the cell signature score associated with the abundance of at least one cell type in said biological sample, relative to the reference value or the cell signature score determined previously, is indicative of the responsiveness of the subject to the treatment.

22. The method of claim 1, wherein said method is a computer-implemented method.

23. A method to identify at least one gene expression signature highly specific for a given cell type, the method comprising:
i) compiling a repertoire of candidate genes for said cell type from a previously published consensus signature and/or a public database,
ii) filtering the candidate gene repertoire for lowly expressed and highly variable genes by comparing the expression levels in the organ of interest, setting a threshold to retain the reliably measurable genes,
iii) clustering the genes based on their correlation on at least three public and/or private datasets and selecting highly correlated gene clusters, in each dataset,
iv) confirming the specificity of the selected gene clusters of each dataset by functional analysis,
v) identifying a core gene signature defined as the gene overlap among the gene clusters selected in each dataset, and
vi) validating the specificity of the gene signature for the target cell type on an independent gene expression dataset derived from the purified or enriched target cell type.

24. The method of claim 23, wherein the gene signature consists in a transcriptomic signature and the threshold corresponds to about 5 transcripts per million (TPM).

25. A device for performing a method according to claim 1 said device comprising:
i) a sample chamber for a test sample collected from a subject;
ii) an assay module in fluid communication with said sample chamber, said assay module comprising means and/or reagents for detecting and/or measuring, directly or indirectly, the gene expression in said test sample;
iii) means for computing a cell signature score; and
iv) a user interface wherein said user interface relates the cell signature score to detecting a disease in said subject, stratifying a disease or determining the responsiveness to a treatment.

26. (canceled)
